(19) United States
(12) Patent Application Publication
(10) Pub. No.: US 2007/0093727 A1
(43) Pub. Date: Apr. 26, 2007

(54) Cervical tissue biopsy system and methods of use

(75) Inventors: Gerald Feuer, Atlanta, GA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSPEC LLC, San Francisco, CA (US)

(21) Appl. No.: 11/259,600

(22) Filed: Oct. 25, 2005

(51) Int. Cl. A61B 10/00

(52) U.S. Cl. 600/564; 600/562; 600/570

(57) ABSTRACT

A cervical tissue sampling system is provided that includes a tissue cutting device and a snare. The tissue cutting device features a conical tip coupled to an elongated body and having a tapered helical cutting edge that defines a tissue receiving channel. The snare includes an elongated body, an adjustable wire loop and a stopper, the stopper arranged to lock the wire loop with a desired degree of tension around the cervix to assist in positioning the tissue cutting device.

### FIELD OF THE INVENTION

The present invention relates to a surgical device for obtaining tissue samples and, in particular, to a system for obtaining cervical tissue samples.

### BACKGROUND OF THE INVENTION

Cervical cancer is a disease having serious consequences, but may be undetected during its early phases. Normally, the disease progresses slowly so procedures for early detection and prevention are available. For example, routine pap smear tests and pelvic exams are performed to detect early signs of the disease.

Detection of an abnormality in the cervix by one of the foregoing techniques indicates additional testing and cervical biopsies are often required. During a cervical biopsy, a tissue sample is taken from the cervix and subsequently examined. Such cervical biopsies may include a cervical “punch” biopsy, wherein small samples of tissue are taken from the cervix, or cone biopsies where a large area of tissue surrounding the external orifice of the cervix is excised.

Biopsies may be performed by hand with a scalpel. Where the procedure is performed by hand, it requires great skill and a steady hand in order to carefully remove the desired tissue.

In an attempt to improve the consistency and safety of biopsy procedures, several devices have been designed specifically for collecting tissue samples. One example of a device for performing a cervical biopsy is described in U.S. Pat. No. 6,730,085 to George et al. The device described in that patent has a shaft including two arms that extend laterally outward a short distance from the distal end of the shaft. An electrically conductive cutting wire extends from the distal end of the shaft to each of the arms, forming a triangular cutting head. In use, the distal end of the shaft is inserted into the cervix so that the wires contact the cervix and a current is passed through the cutting head while the device is rotated to excise a tissue sample. The device is relatively complicated, requiring an electrosurgical generator to supply current to the cutting head. Additionally, the tissue sample may be partially burned during excision, thereby reducing the useful size of the sample.

Another example of a previously-known device for obtaining tissue samples is described in U.S. Pat. No. 4,054,127 to Milan et al. The device described in that patent is designed to scrape a tissue-sample from the endometrial cavity of a woman. The device includes a handle having a spiral member. The spiral member has straight sides and has a curved longitudinal axis that is designed to match the curve of the endometrial cavity. Upon rotation of the handle, the spiral member scrapes the walls of the cavity collecting mucous, cells, and tissue fragments. The patent also describes a slotted paddle for removing the tissue sample from the spiral member. The scraping device is limited to scraping a side wall of a cavity and is incapable of cutting a tissue sample from the wall of the cervix.

Additionally, the cervix is a pliable organ which often makes inserting a biopsy device, such as those described above, difficult. Various gripping devices and snares have been designed to enable a user to grip pliable a organ or tissue. One such device, described in U.S. Pat. No. 5,084,054 to Bencini et al., includes a support assembly and a slide-mounted snare. A gear train coupled to the slide tightens the loop snare. The device does not allow the user to lock the snare with a desired tension, but instead requires the user to manually maintain tension in the snare during use.

In U.S. Pat. No. 4,493,320, the cutting and gripping functions are combined. The device described in that patent includes an insulative tubular member, an actuator and a pair of electrically conductive wires coupled at a distal end to form a snare. The wires are fixedly coupled to the slide block, which is moved relative to the tubular member to tighten the snare. As in the device described in Bencini et al., the snare cannot be locked at a predetermined tension, and in addition, the use of the electrosurgical cutting wire may cause burning of the tissue sample.

In view of the foregoing, it would be desirable to provide a tissue cutting device that precisely cuts tissue samples without damaging the samples.

It further would be desirable to provide a tissue snare that allows the surgeon to lock the tension in the snare to maintain a grip on a pliable tissue or organ.

It also would be desirable to provide a biopsy system capable of obtaining high quality tissue samples while providing the ability to easily locate the cutting tool within pliable tissue.

### SUMMARY OF THE INVENTION

In view of the foregoing, it is an object of the present invention to provide a cutting device that precisely cuts tissue samples, but does not rely upon a cutting modality that can damage the sample tissue.

It is another object of this invention to provide a snare that allows a user to easily grip and control pliable tissue.

It also is an object of this invention to provide a system for obtaining tissue samples that includes a cutting device, a snare and a sample removal device.

These and other objects of the present invention are accomplished by providing a biopsy system comprising a tissue cutting tool and a snare.

In accordance with the principles of the present invention, the tissue cutting tool includes a self-locating conical tip having a tapered helical cutting edge. The conical tip is fixedly coupled to a distal end of an elongated body. A handle disposed at the proximal end of the elongated body transfers rotation from the handle to the conical tip. The handle further includes a tissue removal device that is removably coupled to a proximal end of the elongated body.

The snare includes an elongated body having a lumen, stopper and a wire that extends through the lumen and the stopper to form an adjustable loop at a distal end of the body. The loop is adjusted by pulling the stopper. A portion of the stopper is configured to grippingly engage a proximal end of the elongated body to maintain a desired degree of tension in the loop.

The foregoing tools enable a user to grip and maintain control over the cervix while using the cutting device to remove a portion of the tissue wall. The tissue sample removal device included in the handle permits the sample tissue to be easily removed from the cutting tip. Of course, the cutting device and snare of the present invention may equally well be separately use to biopsy or grip other tissues or organs.

Methods of obtaining tissue samples, for example, cervical tissue samples, also are provided.

### BRIEF DESCRIPTION OF THE DRAWINGS

Further features of the invention, its nature and various advantages will be more apparent from the accompanying drawings and the following detailed description of the preferred embodiments, in which:

FIGS. 1A and 1B are, respectively, side and cross-sectional views of a tissue sample cutting device suitable for use in the tissue sampling system of the present invention;

FIGS. 2A and 2B are, respectively, side and cross-sectional views of a tissue sampling snare for use in the tissue sampling system of the present invention;

FIGS. 3A and 3B are, respectively, side views of the steps in a cervical biopsy procedure using the devices shown in FIGS. 1A, 1B, 2A and 2B; and

FIG. 4 is a side view showing a device for removing a tissue sample from the cutting device of FIGS. 1A and 1B in use.

### DETAILED DESCRIPTION OF THE INVENTION

The present invention is directed to a system for obtaining tissue samples and, in particular, cervical tissue samples. The system of the present invention generally includes a cutting device and a snare for gripping tissue during removal of the tissue sample.

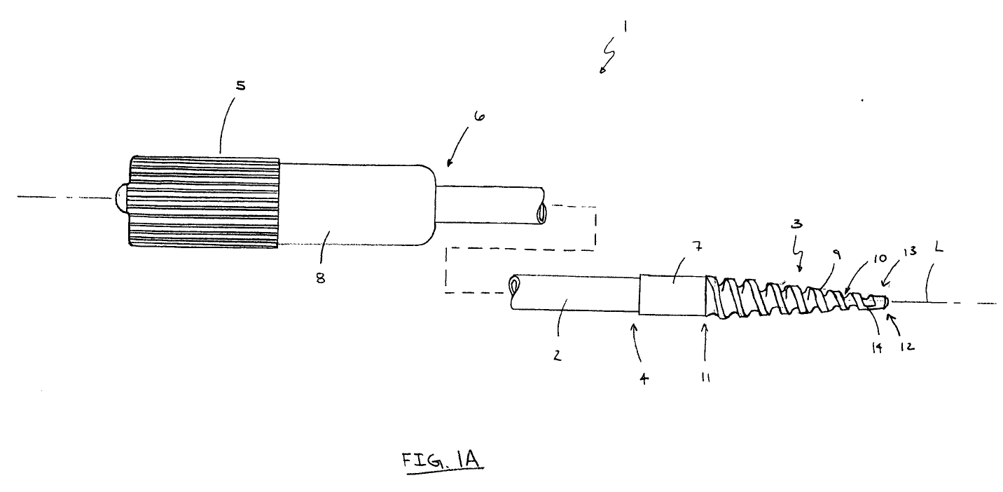

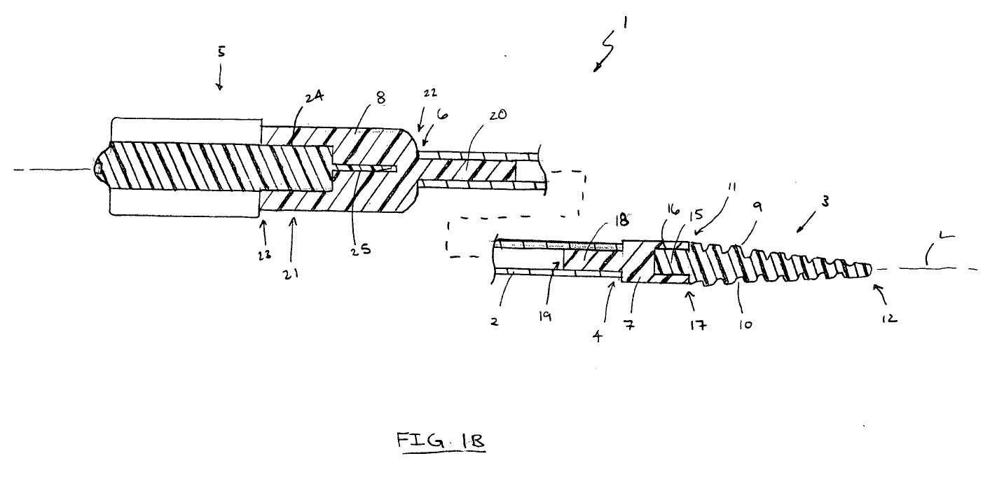

Referring to FIGS. 1A and 1B, an embodiment of tissue cutting device 1, or curette, suitable for use in performing tissue biopsy is described. Cutting device 1 generally includes elongated body 2 and conical tip 3. Cutting device 1 may also include handle 5 coupled to proximal end 6 of body 2. Tip 3 and handle 5 are fixedly coupled to body 2 so that rotational or longitudinal motion of handle 5 is transferred to tip 3. Tip 3 and handle 5 may be coupled directly to body 2 or indirectly (i.e., through respective connectors). In FIGS. 1A and 1B, tip connector 7 and handle connector 8, respectively, are employed to couple tip 3 and handle 5 to body 2.

Conically-shaped tip 3 is designed to cut tissue samples from a body part, such as the cervix, and includes helical cutting edge 9 that defines a helical tissue receiving channel 10. Preferably, the tip has an angle of taper between 2° and 10° with respect to a longitudinal axis of the tip. Tip 3 tapers to a reduced diameter from proximal end 11 to distal end 12. Distal end 14 of cutting edge 9 is spaced from distal end 12 of tip 3 and distal end 12 of tip 3 is rounded to create a self-locating feature 13. Self-locating feature 13 allows cutting device 1 to become seated in the external orifice of the cervix prior to cutting any cervical tissue.

Stud 15 extends proximally from proximal end 11 of tip 3. Stud 15 is configured to engage tip connector 7 and to resist relative rotation between tip 3 and tip connector 7. In a preferred embodiment, tip stud 15 has a square cross-section and tip 3 and stud 15 are monolithic. Alternatively, stud 15 may have any other cross-section suitable to resist relative rotation between tip 3 and tip connector 7, such as a polygonal or star-shaped cross-section. Tip 3 and stud 15 may be constructed from any biocompatible material known in the art. For example, tip 3 and stud 15 may be constructed from polycarbonate plastic or surgical stainless steel.

Stud 15 and tip connector 7 may be coupled by any method known in the art. For example, the method of coupling may include an adhesive, a press or shrink fit, welding (ultrasonic or conventional), brazing and/or soldering or by threads.

As shown, tip connector 7 is generally cylindrical and includes bore 16 at distal end 17 configured to receive stud 15. Bore 16 is preferably shaped so that it has a cross-sectional shape complimenting the cross-sectional shape of stud 15. Tip connector 7 also includes tip connector stud 18 at proximal end 19. Tip connector stud 18 is configured to mount in body 2.

Tip connector 7 may be constructed from any biocompatible material known in the art. For example, tip connector 7 may be constructed from acrylonitrile budadiene styrene (ABS) plastic or surgical stainless steel.

Tip connector 7 is rigidly affixed to body 2 so that there is no relative rotation between these components. Tip connector 7 may be coupled to body 2 using any coupling technique known in the art, such as using threads, an adhesive, a press or shrink fit, welding (ultrasonic or conventional), brazing and/or soldering.

Body 2 is an elongated member that transmits rotational force from handle 5 to tip 3. Body 2 is tubular and a longitudinal bore of body 2 is configured to receive tip connector stud 18 at distal end 4 of body 2. Body 2 may be constructed from any biocompatible material known in the art, such as polyether block amides, e.g., PEBAX (a registered trademark of Ato Chimie of Hauts-de-Seine, France) or surgical stainless steel. Body 2 may be tubular or solid and may be extruded, molded and/or machined to provide the desired dimensions.

Handle connector 8 is coupled to proximal end 6 of body 2. Handle connector 8 is generally cylindrical and includes handle connector stud 20 at distal end 22, and handle receiving portion 21 at proximal end 23. Handle receiving portion 21 includes handle stud receiving bore 24 and spade receiving slot 25. Stud receiving bore 24 is generally cylindrical and spade receiving slot 25 is sized to closely compliment the cross-section of a spade 28 included on handle 5, as described below.

Handle connector 8 may be constructed from any biocompatible material known in the art. For example, handle connector 8 may be constructed from acrylonitrile budadiene styrene (ABS) plastic or surgical stainless steel. Handle connector 8 may be coupled to body 2 using any coupling technique known in the art, such as using threads, an adhesive, a press or shrink fit, welding (ultrasonic or conventional), brazing and/or soldering.

Handle 5 includes grip portion 26, handle stud 27 and spade 28. Grip portion 26 includes features 29 such as ribs, facets, knurling or other texturing, that enhance the user's grip. Handle stud 27 extends distally from grip portion 26 and spade 28 extends further distally from handle stud 27.

Spade 28 is provided to remove a tissue sample from helical tissue receiving channel 10 of tip 3. Spade 28 is configured to fit within a recess of handle 5 and illustratively has a rectangular cross-section. A distal portion of spade 28 is tapered to form a wedge that ends at distal edge 29. Spade 28 is configured so that the wedge at its distal end may be inserted into tissue receiving channel 10 to gently urge a tissue sample out of the channel. Distal edge 29 may be shaped to match the shape of channel 10, for example distal edge may be rounded to match the curvature of channel 10.

Handle 5 may be constructed from any biocompatible material known in the art, such as described above for handle 8. Handle 5 may be removably inserted into handle receiving portion 21 of handle connector 8 so that spade 28 is slidably received within slot 25. In addition, the cross-section of spade receiving slot 25 preferably compliments the cross-section of spade 28. In this manner, when spade 28 is received within slot 25, relative rotation between handle 5 and handle connector 8 is prevented.

Alternatively, grip features may be directly incorporated into body 2, thereby obviating the need for handle 5. In such an embodiment, grip features such as ribs, facets and/or knurling may be incorporated on the exterior surface of body 2. In addition, spade 28 may constitute a separate device rather than being incorporated into handle 8 of the cutting device.

Still further, it should be appreciated that tip 3 and/or handle 5 may be directly coupled to body 2, obviating the need for additional parts such as tip connector 7 and handle connector 8. In this case, tip 3 and handle 5 may be affixed to body 2 using any suitable means, such as threads, an adhesive, a press or shrink fit, welding (ultrasonic or conventional), brazing and/or soldering.

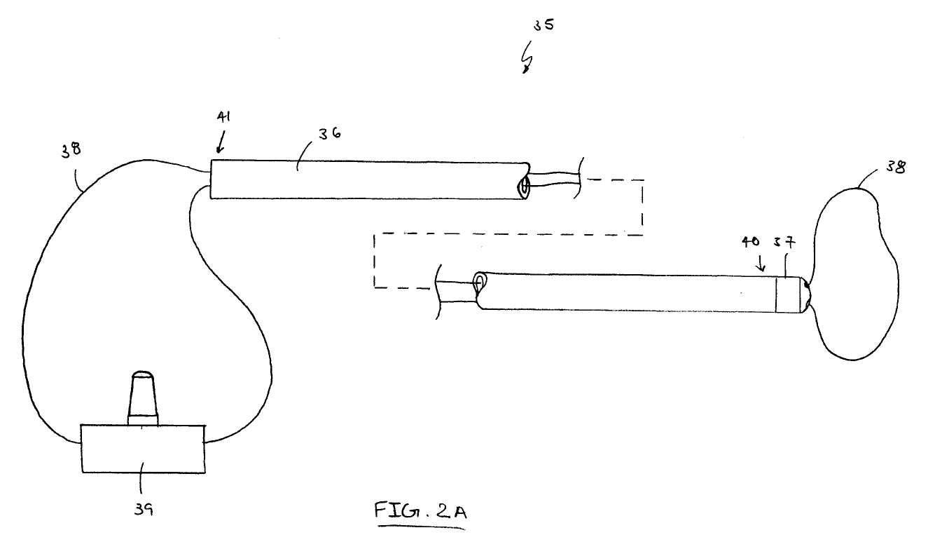

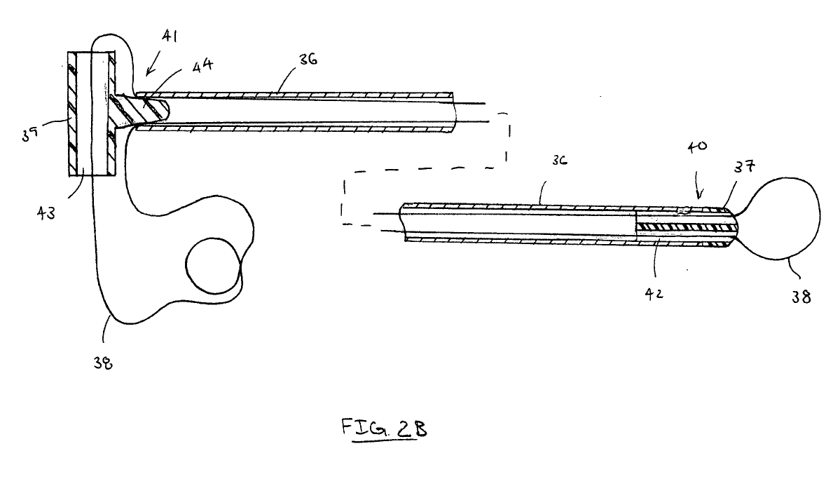

Referring now to FIGS. 2A and 2B, snare 35 suitable for use in the tissue sampling system of the present invention is described. Snare 35 generally includes elongated body 36, tip 37, wire 38 and stopper 39. Body 36 illustratively comprises a tubular member having distal end 40 and proximal end 41, and may be constructed from any biocompatible material known in the art, such as PEBAX or surgical stainless steel.

Tip 37 is coupled to distal end 40 of body 36 and includes bores 42. Bores 42 extend through the full length of tip 37 and communicate with the longitudinal bore of body 36 and the environment. Tip 37 may be constructed from any biocompatible material known in the art, such as ABS plastic or surgical stainless steel.

Wire 38 forms a loop that extends through body 36, bores 42 of tip 37, and stopper 39. Although tip 37 is not necessary for the proper operation of snare 35, the path of wire 38 through bores 42 assures that the wire is not inadvertently pulled proximally out of the device. In addition, tip 37 provides a clamping surface for tissue gripped within the loop of wire 38. Wire 38 may comprise any suitable biocompatible material, such as nylon, silk or polypropylene.

Stopper 39 has wire receiving portion 43 including a lumen and plug 44. A proximal portion of wire 38 extends through the lumen of wire receiving portion 43. Wire receiving portion 43 may be sized so that it fits easily within a user's hand and may be used to manipulate wire 38 during tensioning.

Plug 44 extends perpendicularly from wire receiving portion 43. In a preferred embodiment, plug 44 is conical with the larger diameter portion located adjacent wire receiving portion 43. Plug 44 is configured so that tip 45 has a smaller diameter than the internal diameter of body 36 and the larger diameter portion has a diameter larger than, or approximately equal to, the internal diameter of body 36. Stopper 39 may be constructed from any biocompatible material known in the art, such as ABS plastic or surgical stainless steel.

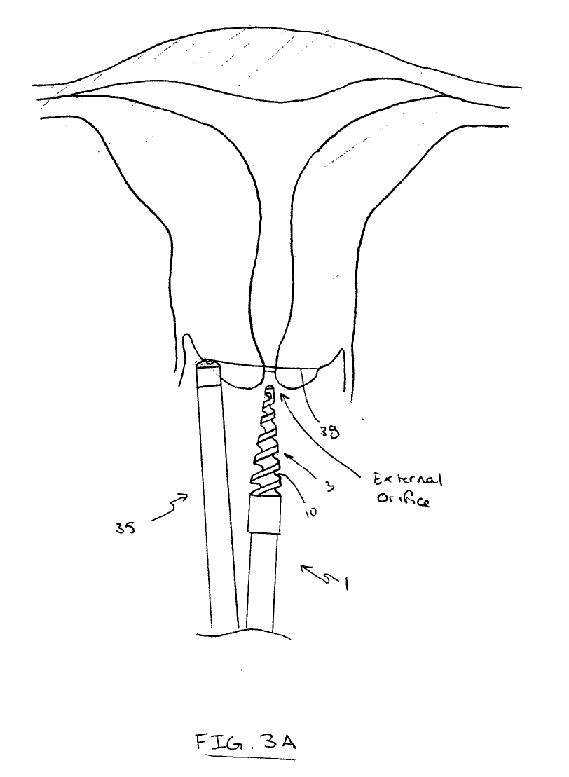

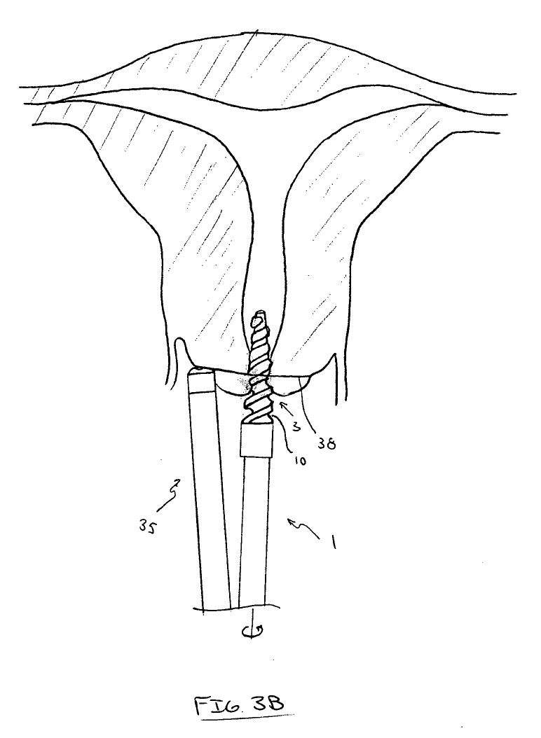

Snare 35 permits the user to capture and retain the cervix in a desired position to simplify the insertion of cutting device 1. In use, wire 38 is extended distally from tip 37 to form a loop. Next, body 36 is manipulated so that the loop of wire is positioned to surround the lower extremity of a patient's cervix that includes the external orifice of the cervix. After wire 38 is so positioned, stopper 39 is retracted proximally with respect to body 36, thereby retracting wire 38 to tighten around the lower portion of the cervix, as depicted in FIGS. 3A and 3B. Once the loop has been reduced enough to give a desired amount of control over the position of the cervix, wire 38 is held stationary with respect to body 35 and plug 44 is inserted into body 36.

The taper of plug 44 assures that wire 38 is wedged between plug 44 and body 36, thereby locking the position of wire 38 with respect to body 36, as shown in FIG. 2B. As a result, wire 38 remains tightened around the cervix without requiring further manipulation by the user and the external orifice of the cervix may be easily accessed.

Referring now to FIGS. 3A and 3B, cervical tissue samples may be removed from the cervix using cutting device 1 in conjunction with snare 35. Snare 35 first is inserted through the vagina and positioned so that wire 38 surrounds the lower extremity of the cervix. Snare 35 then tightened to grip the cervix, as described above, to control the position of the external orifice.

After snare 35 has engaged the cervix and the external orifice is positioned as desired, cutting device 1 may be inserted into the vagina so that distal end 12 of tip 3 is positioned below the cervix in alignment with the external orifice. Tip 3 of cutting device 1 then is inserted into the external orifice of the cervix. Cutting device 1 then may be rotated so that cutting edge 9 excises a continuous sample of tissue, which is received within tissue receiving channel 10. After a sufficient tissue sample has been obtained, cutting device 1 is retracted proximally with the sample disposed within tissue receiving channel 10. Snare 35 may be removed by removing stopper 39 from body 36 to release the tension on wire 38; snare 35 then is removed from the patient's body.

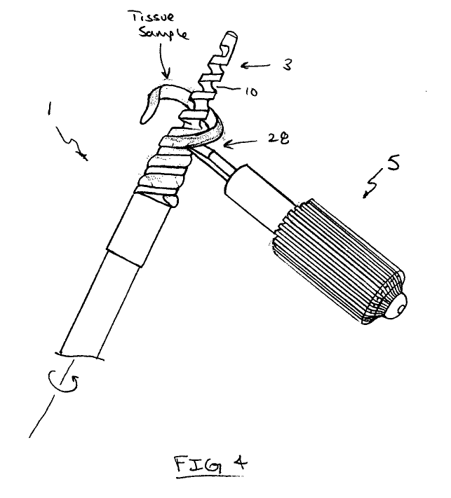

Upon removal of cutting device 1, the tissue sample is held within tissue receiving channel 10. To remove the tissue sample from channel 10, handle 5 may be removed from handle connector 8. Distal edge 29 of spade 28 is inserted into channel 10 between tip 3 and an end portion of the tissue sample. Next, cutting device 1 is rotated while spade 28 is held stationary within channel 10, whereby the spade urges tissue sample out of channel 10, as depicted in FIG. 4. The tissue sample may then be placed in a suitable package to await testing.

Advantageously, the cutting device and snare of the present invention my be manufactured of low-cost polymers, and thus suitable for disposal after a single use. In addition, because polymers generally have a much lower thermal conductivity, the above-described devices are not expected to cause a significant degree of patient discomfort during the biopsy procedure. Alternatively, more durable materials, such as stainless steel, may be used to manufacture the cutting device and snare, in which case the devices may be sterilizable to permit repeated use.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. A cervical tissue sampling system comprising:
an elongated body having proximal and distal ends;
a conical tip coupled to the distal end, the conical tip having a tapered helical cutting edge that defines a tissue receiving channel; and
a handle coupled to the proximal end,
wherein rotation of the handle is communicated to the conical tip.

2. The tissue sampling system of claim 1 further comprising a snare configured to engage a patient's cervix, the snare comprising:
an elongated body having proximal and distal ends;
an wire slidably extending through the body to define proximal and distal loops; and
a stopper disposed on the proximal loop and configured to selectively engage the proximal loop with the patient's cervix, the stopper including a plug selectively engagable with the elongated body to lock the wire loop in a position tightened around the patient's cervix.

3. The tissue sampling system of claim 1 further comprising a tissue sample removal device.

4. The tissue sampling system of claim 3, wherein the tissue sample removal device is received within a recess in the handle.

5. The tissue sampling system of claim 1, further comprising a handle connector interposed between the handle and the elongated body.

6. The tissue sampling system of claim 1, wherein the handle includes grip features.

7. The tissue sampling system of claim 1, further comprising a tip connector interposed between the wherein the conical tip and the elongated body.

8. The tissue sampling system of claim 1, wherein the tip has an angle of taper between 2° and 10° with respect to a longitudinal axis of the tip.

9. The tissue sampling system of claim 1, wherein the tissue cutting device body is tubular.

10. The tissue sampling system of claim 1, wherein the elongated body is made of one of a polyether block amide plastic and stainless steel.

11. The tissue sampling system of claim 1, wherein the conical tip is made of polycarbonate plastic.

12. A cervical tissue biopsy device, comprising:
an elongated body having proximal and distal ends; and
a conical tip coupled to a distal end, the conical tip having a tapered helical cutting edge.

13. The tissue cutting device of claim 12, further comprising:
a handle coupled to a proximal end of the body.

14. The tissue cutting device of claim 13, wherein the handle is removably coupled to the body.

15. The tissue cutting device of claim 14, wherein the handle includes a sample removal device.

16. The tissue cutting device of claim 15, wherein the sample removal device is a spade.

17. The tissue cutting device of claim 13, wherein the handle includes a grip feature.

18. The tissue cutting device of claim 13, further comprising a handle connector interposed between the handle and the elongated body.

19. The tissue cutting device of claim 12, further comprising a tip connector interposed between the conical tip and the elongated body.

20. The tissue cutting device of claim 12, wherein the tip and the elongated body are coupled by at least one of: threads an ultra-violet sensitive adhesive, ultrasonic welding, welding, soldering and brazing.
